(19) United States
(12) Patent Application Publication
(10) Pub. No.: US 2002/0169357 A1
(43) Pub. Date: Nov. 14, 2002

(54) Method and device for the control of appetite

(76) Inventor: Yu Chen, Baltimore, MD (US)

Correspondence Address: SAM Silverberg, 6820 32ND St. NW, WASHINGTON, DC, 20015, US

(21) Appl. No.: 09/853,066

(22) Filed: May 11, 2001

(51) Int. Cl. A61N 1/00

(52) U.S. Cl. 600/15

(57) ABSTRACT

A novel method and device to control appetite, reduce food craving, reduce excess food consumption and treat other eating disorders is provided by attaching or implanting one or more magnets on the acupoints or areas of the ears of human subjects or animals. The device of the invention includes magnets preferably of a circular configuration, having magnet strength of about 100-14,000 gauss. A magnet is attached on an auricular acupoint or area by adhesive plaster. The magnets, adhesive plasters, an illustration, and instruction will be packed in a kit.

### FIELD OF THE INVENTION

The present invention relates to the use of magnets for the treatment of eating disorders such as lack of appetite, food craving, and excess food consumption. The magnets are placed at acupoints areas on ears to achieve the desired effects.

### BACKGROUND OF THE INVENTION

The art of acupuncture has been practiced in China for generations. Acupuncture is the puncturing of the skin with needles at specific points on the body. These points are known as acupoints. The acupoints are associated with meridian channels. The Chinese also developed micro-acupuncture such as auricular acupuncture, scalp acupuncture, and hand and foot acupuncture. Representative acupoints related to each part of the body can be found in the ear. By puncturing these points, the related part of the body receives a signal to have the desired functional effect.

Prior art experiments have shown that electrical stimulation in the lateral hypothalamus can induce eating behavior and causes refusal of food. This area is known as the hunger center. Whereas electrical stimulation in certain ventromedial regions of the hypothalamus inhibits eating behavior elicit excessive eating and obesity. This is known as the satiety center.

The micro-injection of noradrenaline into the hypothalamus is known to dramatically increase food intake. Direct injection of serotonin into hypothalamus suppresses food intake, and drugs that release serotonin from nerve endings decrease food intake and body weight. The higher centers of the brain are also involved in eating and drinking.

Medical treatment using magnets is found in Chinese history. In “Magnetic Therapy” written by Dr. Chen Zhi, the Chinese expert on magnet therapy, teaches using magnet on the body system such as trunk, leg, and arm etc. In the prior art, pain has been treated by the use of magnets. For example, Australian Patent AU-B-30256/92 points out that large areas of the outer ear can be exposed to magnet force to stimulate or sedate a person. In contrast, the present invention is directed to the placement of magnets on specific acupoints or areas to control appetite, reduce food craving, and other eating disorders.

Acupuncture for controlling appetite and reducing food craving is by needling the acupoints or giving pressure on the acupoints. Using needles to puncture ears is not acceptable by many people and the effect will not last long. It is not practical. The acupuncture and acupressure have to be done by an acupuncturist. The long-time pressure causes ear damage. There is a need to have a simple, non-invasive, self-care, low-cost new method to reduce appetite and control food craving.

### SUMMARY OF THE INVENTION

This invention is directed to the placement of one or more magnets of effective strength on a specific acupoint or acupoints or area(s) to treat eating disorders such as appetite control and reduce food craving.

The magnets are usually with the strength of 100 to 14,000 gauss. Magnetic strength of 1000 to 3000 gauss is preferred. The size and the shape that will be applied to the acupoint(s) or area(s) depends on the location of the acupoint(s) or area(s). The magnet can be a round ball, circular disc, square, rectangle, dome, or other shape, even with decoration. It can be coated with 24 k gold, titanium, niobium, platinum, or any other coating material. The magnet can be polished well without special coating material. In most cases the magnet is stuck to the skin by hypoallergenic adhesive tape. The size of the tape varies according to the size of the magnet. A small magnet needs a small tape; a big magnet needs a big tape. The shape of the tape can be varied. U.S. Pat. No. 5,803,896 to this inventor points out various ways to attach a magnet to an ear.

The magnet can also be implanted in the auricle acupoint. This method and device are for human being and other animals.

### BRIEF DESCRIPTION OF THE DRAWINGS

FIG. 1. is a side view of the anatomy of the left ear of a human subject,

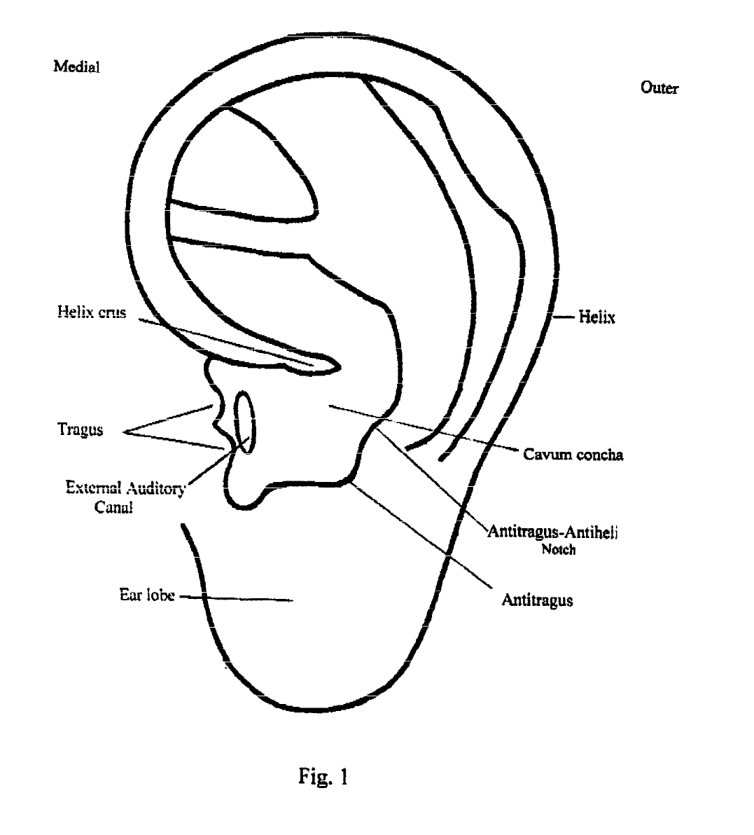

FIG. 2a shows the auricular point and area locations.

FIG. 2b shows the auricular point and area locations.

### DETAILED DESCRIPTION OF THE INVENTION

The objective of this invention is to place a magnet(s) on auricular acupoint(s) or area(s) to treat food disorders. The auricular acupoints used are the Thalamus, Exciting point, Hunger point, Spleen, Esophagus, and San Jiao. The areas are the hypothalamus area and the stomach area.

The present invented therapy is a simple method to control appetite or craving of food. Adequate physical exercise is equally important to increase the expenditure of caloric intake. Choosing acupoints in the ear also stimulates the person to do more physical movement including exercise.

A. The Choice of Acupoints and Areas:

1. Thalamus:

Thalamus is the first choice for controlling appetite and reducing craving of food. The acupoint, Thalamus, here corresponds to hypothalamus. This acupoint is important for food ingestion. In most cases, the placement of a magnet on this point is sufficient to treat eating disorders, such as food craving, excess consumption and loss of appetite. The Thalamus point can be used alone or in combination with the other set forth acupoint(s) and area(s).

2. Exciting Point:

This point has the effect of stimulating physical exercise thereby having a positive effect on eating disorders. This acupoint can not be used alone and should be used in combination with the other set forth acupoint(s) or area(s). Because of its proximity to the Thalamus point, a magnet that covers both points can be used.

3. Hunger Point:

This point is used for controlling appetite and can be used for treating obesity, hyperthyroidism, and nervous overeating. It is a key point for reducing appetite. This acupoint can be used alone or in combination with the other set forth acupoint(s) or area(s).

4. Spleen:

This point has the function of improving metabolism and reducing abnormal food craving. This acupoint can be used alone or in combination with the other set forth acupoint(s) or area(s).

5. San Jiao:

This point improves metabolism and reduces abnormal food craving. This acupoint can be used alone or in combination with the other set forth acupoint(s) or area(s).

6. Esophagus

This point treats digestive problems. This acupoint can be used alone or in combination with the other set forth acupoint(s) or area(s).

7. Hypothalamus Area;

This area is important for ingestion. A magnet placed in this area can correct eating disorders. This area can be used alone or in combination with other acupoint(s) and stomach area.

8. Stomach Area:

A magnet placed in this area can effect food intake and reduce food craving This area can be used alone or in combination with other acupoint(s) and Hypothalamus area.

When large and strong magnets are used, it is better to choose fewer points to avoid the magnets attraction to each other. Or choose two points that are far enough from each other to avoid magnets attraction to each other. For example, use one magnet disc that covers both thalamus and exciting point, or use a small magnet ball attached to the hunger point.

The locations of the acupoints are described below:

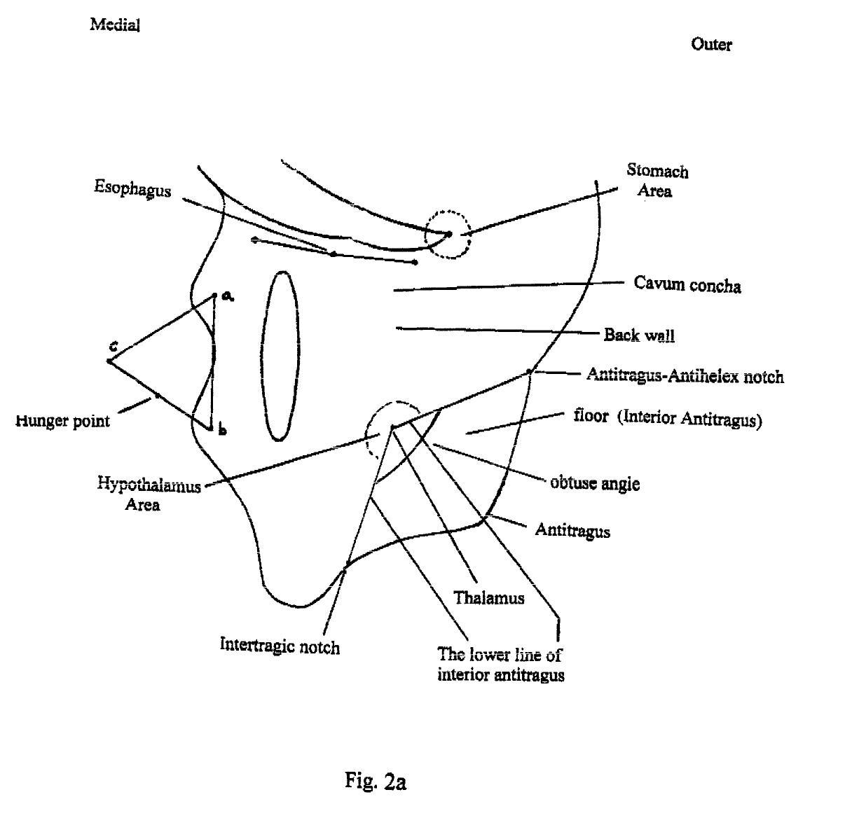

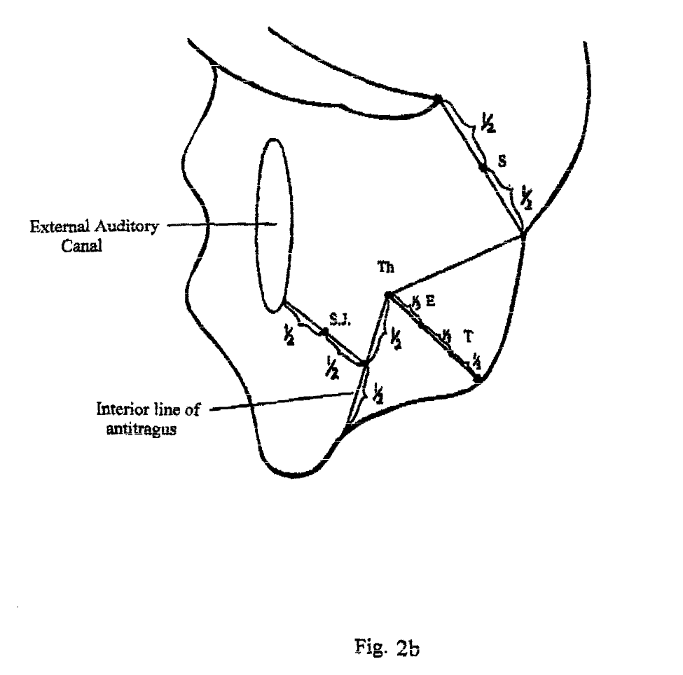

1. FIGS. 2a and b show the Thalamus (Th) point located at the mid-point of the lower line of the interior antitragus. The lower line of the antitragus is in the bottom of the Cavum Concha. The Cavum Concha can be described as a cave, the back wall is vertical and the bottom floor is horizontal. This bottom floor is the interior antitragus. The connection of the back wall and the floor is the lower line of the antitragus. This line is not a straight line but a line with an obtuse angle. The opening of this angle is toward the antitragus. The beginning point of this line is the intertragic notch and the endpoint of the line is the antitragus-Antihelix notch. The mid-point of this line is the Th point. The angle is usually 100-140 degrees.

2. FIG. 2b shows the Exciting Point (E) located on a line between Thalamus (Th) point and the top of the antitragus (P). The Exciting Point (E) is located by dividing the line into three equal parts. E is one third down on the line from Th going toward P.

3. FIG. 2b shows the Hunger point (H) located at the middle of the line bc. The line bc can be determined by dividing the trigus into two parts, the upper part and the lower part. The center of the upper part is a, the center of the lower part is b. The line from a to b forms one leg of an equilateral triangle. The third point c of this triangle is toward the medial (face) and located on a perpendicular line intersecting half way between ab. The middle point of be is Hunger point (H). To be effective there is a need to locate the hunger point accurately. At this point, a small magnet ball or small magnet disc with a diameter not bigger than 0.15 cm should be applied. If a larger magnet is used, other acupoints will be covered that in combination might cause increased appetite.

4. FIG. 2b shows the Spleen (S) located at the midpoint of a line from the end of Helix crus to Antitragus-Antihelix notch.

5. FIG. 2b shows the San Jiao (S.J.): located at the middle area from the inferior and lateral edge of the hole of the External Auditory Canal to the interior line of the antitragus.

6. FIG. 2a shows the Hypothalamus area is the area that surrounds the Thalamus point. This area is about 0.3 cm in diameter.

7. FIG. 2a shows the stomach area. This area is located at the area where the Helix Crux disappears. The diameter of this area is about 0.4 cm.

8. FIG. 2a shows the esophagus located at about the middle ⅓ part inferior to the Helix Crux.

All the above points can be selected for reducing appetite. Usually the Thalamus point, is sufficient for reducing appetite and to decrease food craving. Since Exciting point is very close to Thalamus, when a magnet disc is applied, both Thalamus and Exciting point can be covered under a single disc. Other points can be chosen in addition to Thalamus and Exciting point. As long as the magnet discs are small enough and do not attract each other to change the position of the magnets, many points can be used. A large magnet disc with high magnet strength might effect the position of another magnet.

The magnetic disc does not have to be centered on the acupoint or area to be effective. For example a magnet disc with diameter of 0.5 cm, can be off 0.15 cm from the center of the point or area. Of course the greater the distance from the point or area the less effective is the treatment. In most of the cases, both ears should be used for attaching magnets.

The size and the effective strength of the magnet can very depending on the patient. The magnet strength of the disc or pellet is 100-14,000 Gauss. The preferred magnet strength is 1000-3000 Gauss. The shape of the magnet can be circular disc, round ball, or oval, square, rectangle or dome. The diameter of the magnet disc or ball is 0.1-0.7 cm, preferred 0.1-0.5 cm. The thickness for the magnet disc is 0.1-0.3 cm. The magnet disc or pellet can be coated with 24 k gold, platinum, niobium, titanium, or other non-poisonous coating. A well-polished surface can be used without coating. The preferred magnets are round discs 0.5 cm in diameter and 0.3 cm thick with 24 K gold coating or polished smoothly. For Hunger point, a 0.15 cm magnet pellet or disc is suitable.

Hypoallergenic adhesive tape can be used for attaching a magnet on the ear. It can be any shape and any color. It is preferred circular shape and tan color. The size of the tape depends on the size of the magnet. A big magnet needs a big tape; a small magnet needs a small tape, as long as the tape can keep the magnet staying in the position securely.

Attaching the magnets to the acupoint(s) or area(s) by using an adhesive tape can treat appetite. The magnets can be applied to both ears. The magnets can be kept on the ear(s) for one week. Once a week the tapes and magnets should be removed. The ears should be washed. After several days or hours the treatment can be resumed.

The following cases show the effects of the treatment.

Case 1. A 33 year-old lady used to eat 1 lb. of chocolate every day. After using a 2500 Gauss magnet on Thalamus and Exciting point in both ears, she stopped eating chocolate.

Case 2. A 52 year-old lady craved doughnuts. She ate 7-8 doughnuts a day. If she was driving on the highway, she had to pull off from the highway in order to buy doughnuts. After using a 2500 Gauss magnet discs on Thalamus and Exciting point on both ears, she stopped eating doughnuts, and her appetite was reduced 50%.

Case 3. A 90 year-old man lost his appetite. He could only eat ¼ of his usual amount of food. After applying 1000 Gauss magnets on Thalamus on ears, his appetite came back to normal.

Case 4. A 43 year-old woman got a diagnosis of cancer. She lost her appetite. By using 1000 Gauss magnets on Thalamus on both ears, her appetite recovered

Case 5. A 35 years old man had an accident in his left foot. As a result part of the left foot was amputated. After surgery he had had foot pain for 5 years and developed depression and lost his appetite. The stress and the antidepressor made him eat more and gain weight. After using 3000 Gauss magnets on Thalamus point on both ears, his appetite reduced 30%.

Case 6. A 50 years old lady had high appetite. She tends to eat “wrong food” such as potato chips, chocolate, or crackers. After magnets with 3000 Gauss were placed on San Jaio on both ears, her craving for food stopped.

Case 7. A retired 68 years old diabetic man craved food. After placing one magnet disc with 3000 Gauss strength on Spleen on left ear. His craving of food stopped became normal.

Case 8. A fifty years old woman complained that she think about food almost all the time. Her regular three meals schedule was disturbed by hunger and craving of food. After using a small magnet ball on hunger points on both ears, her abnormal hunger and craving were alleviated.

Case 9. A 28 year old woman with high appetite and a craving for sweet food was treated by placing a 3000 gauss magnet disc on her stomach area of left ear and a 3000 gauss magnet disc on her esophagus point of right ear. Her appetite and food craving were reduced.

Case 10. A 27-year-old man showed a reduction of appetite after treatment with a 3000 gauss magnet on the hypothalamus area.

1. A method for the alleviation of eating disorders comprising the placement of one or more magnets having an effective strength on one or more auricular acupuncture points or areas of one or both ears.

2. A method of claim 1 wherein the eating disorders are excess appetite or lack of appetite or craving for food, or excess consumption of food.

3. The method of claim 1 wherein the placement is on the Thalamus point.

4. The method of claim 1 wherein the placement is on the Exciting point.

5. The method of claim 1 wherein the placement is on the Hunger point.

6. The method of claim 1 wherein the placement is on the Spleen point.

7. The method of claim 1 wherein the placement is on San Jiao point.

8. The method of claim 1 wherein the placement is on esophagus point.

9. The method of claim 1 wherein the placement is in the hypothalamus area.

10. The method of claim 1 wherein the placement is in the stomach area.

11. The method for the control of appetite and reduce craving of food of claim 1 wherein said magnet is circular and has a magnetic strength of from about 100 to 14,000 Gauss and said magnet is a hypoallergenic magnet.

12. The method for the control of appetite and reduce craving of food of claim 11 wherein said preferred magnet strength are 1000 to 3000 Gauss.

13. The method for the control of appetite and reduce craving of food of claim 11 wherein said hypoallergenic magnet is 24 k gold plated.

14. The method for the control of appetite and reduce craving of food of claim 11 wherein said hypoallergenic magnet is a magnet with platinum or titanium coating.

15. The method for the control of appetite and reduce craving of food of claim 11 wherein said hypoallergenic magnet is a magnet with well-polished surface.

16. The method for the control of appetite and reduce craving of food of claim 11 wherein said hypoallergenic magnet is a magnet having an inert material interface.

17. The method for the control of appetite and reduce craving of food of claim 1 wherein said magnet is placed on an auricular acupuncture point by hypoallergenic plaster.

18. The method for the control of appetite and reduce craving of food of claim 1 wherein said magnet is placed on the auricular acupuncture point by implantation.

19. The method for the control of appetite and reduce craving of food of claim 1 wherein the ear is human or animal ear.

20. A kit for controlling appetite and reducing craving of food comprising:
(a) an illustration of at least one acupoint or area location in the auricle of the ear;
(b) plurality of magnets having a strength of from 100 to 14,000 Gauss; and
(c) means for mounting one or more of said plurality of magnets in ears.
(d) an instruction paper

21. The kit for controlling appetite and reducing craving of food of claim 20 wherein said illustration is a plastic model or a drawing of left or right ear illustrating Thalamus point.

22. The kit for controlling appetite and reducing craving of food of claim 20 wherein said illustration is a plastic model or a drawing of the left or right ear illustrating the Exciting point.

23. The kit for controlling appetite and reducing craving of food of claim 20 wherein said illustration is a plastic model or a drawing of the left or right ear illustrating the Hunger point.

24. The kit for controlling appetite and reducing craving of food of claim 20 wherein said illustration is a plastic model or a drawing of the left or right ear illustrating the Spleen point.

25. The kit for controlling appetite and reducing craving of food of claim 20 wherein said illustration is a plastic model or a drawing of the left or right ear illustrating the San Jaio point.

26. The kit for controlling appetite and reducing craving of food of claim 20 wherein said illustration is a plastic model or a drawing of the left or right ear illustrating the esophagus point.

27. The kit for controlling appetite and reducing craving of food of claim 20 wherein said illustration is a plastic model or a drawing of the left or right ear illustrating the stomach area.

28. The kit for controlling appetite and reducing craving of food of claim 20 wherein said illustration is a plastic model or a drawing of the left or right ear illustrating the hypothalamus area

29. The kit for controlling appetite and reducing craving of food of claim 20 wherein said means for mounting one of said plurality of magnet in an ear is an adhesive plaster.

30. A magnet for controlling appetite and reducing craving of food claim 1 comprising:
(a) an implantable magnetic body having a strength of from 100 to 14,000 Gauss: and
(b) constructed of a hypoallergenic material
